(19) United States
(12) Patent Application Publication
(10) Pub. No.: US 2005/0042272 A1
(43) Pub. Date: Feb. 24, 2005

(54) Vesiles derived from t cells, production and uses

(76) Inventors: Yafei Hou, Mountain View, CA (US); Du-Hwei Hsu, Sunnyvale, CA (US); Anita Mehta-Damani, Sunnyvale, CA (US); Henry Lamparski, San Mateo, CA (US); Redro Paz, Hercules, CA (US); Jean-Bernard Le Pecq, Menlo- Park, CA (US)

Correspondence Address: NIXON & VANDERHYE, PC, 1100 N GLEBE ROAD, 8TH FLOOR, ARLINGTON, VA, 22201-4714, US

(21) Appl. No.: 10/502,333

(22) PCT Filed: Mar. 13, 2003
(86) PCT No.: PCT/IB03/01391

Related U.S. Application Data
(60) Provisional application No. 60/363,849, filed on Mar. 14, 2002.

(51) Int. Cl. A61K 9/127; C12P 7/64; A61K 38/20

(52) U.S. Cl. 424/450; 424/85.1; 435/134

(57) ABSTRACT

The present invention relates to compositions comprising vesicles released from activated T lymphocytes, as well as to methods for their production and uses. Said vesicles contain a set of bioactive molecules which confer remarkable properties, such as antigen recognition, antigen presentation and other regulatory and effector functions. This invention also relates to methods for transferring or delivering antigenic molecules (e.g., peptides, peptide/MHC complexes, TCR or subunit thereof, etc.) to antigen presenting cells (APCs) using said vesicles, to induce specific immune responses, particularly specific CTL responses. The invention further relates to methods of delivering molecules selectively or specifically to target cells using said vesicles.

### INTRODUCTION

The present invention relates to compositions comprising vesicles released from activated T lymphocytes, as well as to methods for their production and uses. Said vesicles contain a set of bioactive molecules which confer remarkable properties, such as antigen recognition, antigen presentation and other regulatory and effector functions. This invention also relates to methods for transferring or delivering antigenic molecules (e.g., peptides, peptide/MHC complexes, TCR or subunit thereof, etc.) to antigen presenting cells (APCs) using said vesicles, to induce specific immune responses, particularly specific CTL responses. The invention further relates to methods of delivering molecules selectively or specifically to target cells using said vesicles.

This invention can be used in research, diagnostic and therapeutic areas, particularly for regulating an immune response in a subject, including human subjects.

### BACKGROUND

The immune system is composed of two principal components: leucocytes and soluble mediators. Various leukocyte subsets compose an immune regulatory network in which the maturation and activation of each population is affected by the others. The communications between immune cells occur mainly through two ways: one is the binding of membrane-bound molecules and their ligands by cell-cell contact. The other is based on soluble mediators produced by one cell, that diffuse to bind their receptors on the other cells. A phenomenon is noted that some membrane exovesicles, often referred to as exosomes (60-80 nm in diameter), are released into the extra cellular space from many different cell types, especially from the cells of the hematopeitic lineage. Exosomes from B Cells (Raposo et al 1996), mast cells (Raposo et al 1997), dendritic cells (Zitvogel et al 1998), activated platelets (Heijnen 1999), T cells (Denzer et al 2000), macrophages (Denzer et al 2000), tumor cells (Wolfers et al 2001) and intestinal epithelial cells (Van Niel et al 2001) have been described. Exosomes are formed within endosomes as specific proteins and lipids are recruited into inwardly budding vesicles. They accumulate in a specific cellular compartment, the multivesicular bodies (MVB). When the limiting membrane of the MVB fuses with the plasma membrane, exosomes are released. The release of exosomes is regulated by specific stimulations in several cell types. Exosomes from different origins exhibit discrete sets of molecular moieties (Escola et al 1998, Thery et al 1999, 2001, Clayton et al 2001). Tetraspanin proteins such as CD9, CD81 are present in large number in all exosomes. Exosomes from dendritic cell (DC), a professional APC, are particularly enriched with MHC class I/II molecules. They are strongly immunogenic and can eradicate pre-established tumor in mice (Zitvogel et al 1998). B cells exosomes transfer MHC class II complexes to follicular dendritic cells (Denzer et al 2000). Exosomes can deliver membrane-bound proteins to target cells in long distant without cell-cell contact and permit the exchange of material between cells. The use of exosomes derived from antigen presenting cells for triggering specific immune responses in human beings has been proposed (WO9705900, WO9903499).

T lymphocytes, together with B cells, represent the two antigen-specific components of the cellular immune system. The activation of T cells is critical to most immune responses and allows other immune cells to exert their functions. T cells may be subdivided into two distinct classes: CD4+ T cells and CD8+ T cells. The regulatory function of CD4+ T cells on the target cells such as B cells, dendritic cells, macrophages and other T cell subsets, and the effector function of CD8+ T cells to kill tumor cells or cells infected with intracellular microbes depend both on cell-cell contacts through cell surface molecules and on the wide array of cytokines they secrete when they are activated. Given that exosomes might be a pathway to implement the communication between the immune cells, T cells exosomes could be mediators able to exert specific regulatory or effector functions. However, until now, no biological function was described for these T cells exosomes, and no efficient method of preparing these vesicles in a biologically active state has been reported.

### SUMMARY OF THE INVENTION

The present invention now discloses that T cell-derived exosomes display unexpected composition and biological activities. The invention also provides advantageous and efficient methods for producing, isolating and/or purifying large quantities of T cell derived exosomes that carry various sets of bioactive molecules. These methods, under particular preferred conditions allow a triggering of the release, an increased yield or a change of the properties of said vesicles. The invention also discloses that advantageous vesicles may be produced from T cells of various species, including various T cell subsets, T cell lines, clones, hybridomas, etc. This invention also discloses methods for functionalizing these vesicles, through direct loading thereof or by treatment of the producer cells. The invention further demonstrates that these vesicles can be used to efficiently and/or selectively deliver molecules to target cells and antigen-presenting cells, particularly for producing or regulating an immune response in mammals. (including humans). The methods described in this invention allow the production and the provision of well-defined T cells exosomes with discrete sets of bioactive molecules from various T cell origins for use in the therapeutic or prophylactic areas or as research tools.

An object of the present invention resides more particularly in a method of producing lipid vesicles, the method comprising:
a) culturing a biological preparation comprising T lymphocytes under conditions allowing the release of membrane vesicles from T lymphocytes, and
b) collecting or purifying vesicles produced in a).

The biological preparation may comprise freshly isolated T cells, in vitro expanded T cells, T cell clones, T cell lines or T cell hybridomas. Furthermore, the biological preparation may be enriched in or depleted for particular T cell sub-populations, such as CD4+ cells, CD8+ cells, ?dt cells, NK-T cells, or for NK cells, etc. The cells may also be genetically modified to encode any desired product or activity, or otherwise treated or altered to control their properties.

According to a preferred embodiment, the biological preparation comprises in vitro or ex vivo expanded T cells.

According to an other preferred embodiment, the biological preparation comprises a T cell line.

According to an other preferred embodiment, the T cells are subjected to an activation treatment.

In a further preferred embodiment, the method comprises a step of funtionalization of the vesicles, either prior to, during or after their release by the T cells. Functionalization of the vesicles results in the production of modified vesicles comprising one or several selected molecules. This may be achieved by direct or chimeric loading of molecules on the vesicles, or by modifying the producing T cells (indirect loading).

In this regard, a particular object of the present invention is a method of producing functionalized vesicles, the method comprising:
a) culturing a biological preparation comprising T lymphocytes under conditions allowing the release of membrane vesicles from T lymphocytes,
b) collecting or purifying vesicles produced in a), and
c) contacting said vesicles with a selected molecule under conditions allowing said molecule to interact with the vesicles, so as to produce functionalized vesicles.

The selected molecule may be an antigenic molecule, such as a peptide, protein, lipid, glycolipid, etc. It may also be any molecule such as nucleic acids, enzymes, hormones, small organic molecules, markers, etc. Typical examples include viral proteins or fragments thereof, such as glycoprotein E2 of HCV, as well as chimeric proteins comprising a polypeptide fused to glycoprotein E2 or lactadherin or a variant or fragment thereof.

In a preferred embodiment, the molecule is an antigenic molecule, and said contacting is performed under conditions allowing said molecule to associate with an antigen-presenting molecule (e.g., a MHC molecule) at the surface of the vesicle.

In an other particular embodiment, the molecule is a chimeric molecule comprising a polypeptide or other active moiety fused to lactadherin or a variant or fragment thereof, and said contacting is performed under conditions allowing said chimeric molecule to associate with phosphatidylserine at the surface of the vesicle.

In an other particular embodiment, the molecule is a chimeric molecule comprising a polypeptide or other active moiety fused to glycoprotein E2 or a variant or fragment thereof, and said contacting is performed under conditions allowing said chimeric molecule to associate with CD81 at the surface of the vesicle.

In an other variant (indirect loading), the method of producing functionalized vesicles comprises:
a) culturing a biological preparation comprising T lymphocytes under conditions allowing the release of membrane vesicles from T lymphocytes, the biological preparation comprising T lymphocytes containing a recombinant nucleic acid encoding a selected molecule, and
b) collecting or purifying vesicles produced in a), said vesicles (or some of them at least) comprising said selected molecule.

In an other variant, the method comprises:
a) culturing a clonal population of T lymphocytes having a determined T cell receptor under conditions allowing the release of membrane vesicles from T lymphocytes, and
b) collecting or purifying vesicles produced in a), said vesicles expressing at their surface said specific T cell receptor.
   Such vesicles are capable of targeting cells expressing a specific MHC I or I/II petides complexes at their surface, allowing the targeted delivery of any selected molecule to said cells.

A further object of this invention resides in a method of producing a pharmaceutical composition, the method comprising:
a) culturing a biological preparation comprising T lymphocytes under conditions allowing the release of membrane vesicles from T lymphocytes,
b) collecting or purifying vesicles produced in a), and
c) conditioning said vesicles in a pharmaceutically acceptable carrier or excipient.

A further object of this invention is a pharmaceutical composition comprising a membrane vesicle and a pharmaceutically acceptable vehicle or excipient, wherein said vesicle is obtained from T lymphocytes. More preferably, the vesicle comprises a selected molecule, such as a drug or an antigenic molecule. Most preferably, the vesicle comprises a complex between a MHC molecule present within said vesicle and an exogenous antigenic peptide.

An other object of this invention resides in a method of stimulating an immune response against an antigen in a subject, comprising administering to the subject an effective amount of a composition or vesicle as defined above. More particularly, a method of this invention comprises:
a) culturing a biological preparation comprising T lymphocytes (such as for instance T cell line, autologous T cells or subsets thereof) under conditions allowing the release of membrane vesicles from T lymphocytes,
b) functionalizing said vesicles by contacting the vesicles with an antigenic molecule under conditions allowing said molecule to bind said vesicles, preferably to associate with an antigen-presenting molecule at the surface of the vesicles, making them immunogenic
c) collecting or purifying vesicles produced in b),
d) conditioning said vesicles in a pharmaceutically acceptable carrier or excipient, and
e) administering the vesicles to a subject in an amount effective to stimulate an immune response.

In a variant, the vesicles are functionalized after purification step c).

In an other variant the vesicles are used to sensitize, stimulate or expand immune cells (such as antigen-presenting cells) in vitro or ex vivo, the immune cells being subsequently administered to the subject in need thereof.

An other object of this invention is a method of delivering an antigenic molecule to an antigen-presenting cell, particularly dendritic cells, comprising contacting antigen-presenting cells with a composition or an immunogenic vesicle as defined above, said vesicle comprising said antigenic molecule. Contacting may be performed in vitro, ex vivo or in vivo. For in vivo contacting, the composition or an immunogenic vesicles are administered to the subject in an effective amount to deliver antigenic molecules to APCs upon contacting said cells in vivo.

An other object of this invention is a method of stimulating dendritic cells, comprising contacting dendritic cells with a composition or an immunogenic vesicle as defined above. Contacting may be performed in vitro, ex vivo or in vivo. For in vivo contacting, the composition or an immunogenic vesicles are administered to the subject in an effective amount to deliver antigenic molecules to APCs upon contacting said cells in vivo.

A further object of this invention is a method of delivering a molecule to a target cell, comprising contacting said target cells with a composition or an immunogenic vesicle as defined above, said vesicle comprising said molecule. Delivery is most preferably targeted through specific markers present at the surface of the vesicles, such as ligands, receptors, antigens, etc., or functional fragments or derivatives thereof. In a particular embodiment, targeting is mediated by the specific T cell receptor (TCR) present on the vesicles. In this particular way of performing the present invention, the vesicles carrying the selected (bioactive) molecule(s) can be targeted specifically to the cells expressing the antigenic peptide/MHC complex that is recognized by the TCR.

The molecule may be exposed at the surface of the vesicle, or contained within said vesicle. The molecule may be of various nature and display a wide range of properties or activities. Contacting may be performed in vitro, ex vivo or in vivo. For in vivo contacting, the composition or an immunogenic vesicles are administered to the subject in an effective amount to deliver, said molecules to said cells in vivo.

A further aspect of this invention relates to methods of characterizing a preparation of vesicles derived from T cells, the method comprising:
isolating such vesicles from a biological preparation comprising T lymphocytes, and
determining the quantity or the quality of said vesicles by absorbing the same on a support (e.g., beads, plates, column, etc.) and assessing the presence of specific markers at the surface of these vesicles. Typically, the support is a bead, such as aldehyde-beads (non-specific binding) or specific antibody-coated magnetic beads (specific binding), or a plate, such as a microwell plate. The characterization can be performed by phenotype analysis (e.g., by FACS) or by ELISA (WO01/82958). In a particular embodiment, the vesicles are isolated by subjecting the vesicles or biological preparation to concentration, ultrafiltration, diafiltration and/or ultracentrifugation on gradient.

The present invention also relates to the use of T cell derived vesicles as disclosed above for the manufacture of a pharmaceutical composition for delivering molecules to cells in vivo, in vitro or ex vivo.

### LEGEND TO THE FIGURES

FIG. 1: Phenotype of the membrane vesicles produced by Phytohemagglutinin (PHA)-activated T cells.

FIG. 2: Phenotype of the membrane vesicles produced by the PHA-activated Jurkart T cells.

FIG. 3: Vesicles generated from activated T cells from three different leukopacks, loaded with the superantigen SEE, induce the release of IL2 by Jurkat cells in the presence of APC.

FIG. 4: Direct Loading of vesicles from T cells with HLA-A2-specific peptides.

FIG. 5: Direct Loading of vesicles from T cells with HLA-A2-specific Mart-1 peptide.

FIG. 6: Mart-1 peptide-loaded vesicles induce Mart-1 specific T cell response in the presence of antigen presenting cell

### DETAILED DESCRIPTION OF THE INVENTION

The present invention is based on a series of unexpected observations and findings. Originally, we observed that exosomes purified from the supernatant of cultured human dendritic cells, enriched from peripheral blood, contained a small fraction of vesicles with markers specific of T cells. This suggested that T cells contaminating the culture might also release exosomes. After further investigations, we found that purified T cells cultured with various stimulations were indeed able to produce vesicles that contained functional proteins and other markers specific of T cells. Based on the size, density and morphology under the electron-microscopy (EM) of the membrane vesicles derived from T cells, we concluded that said vesicles possess physical properties resembling exosomes derived from other types of immune cells.

The vesicles produced from. T cells, however, possess particular features and components which confer specific properties. The markers or components displayed by these T cells-derived vesicles can be put under four categories, based on their bioactivities:
antigen recognizing proteins, such as T cell receptor (TCR) and CD8;
antigen presenting proteins, such as MHC class I and class II molecules;
regulatory effector proteins such as CTLA-4 and perforin; and
other T cell proteins and markers with unclear functions.

The present application now provides evidence that T cells of various origins secrete particular vesicles, that the release may be activated or controlled by various treatments, that the vesicles can be isoated in large quantities, that the vesicles display particular sets of molecules having functional structure and bioactivities, and that these vesicles may be further functionalized. For example, the MHC class I/II molecules enriched on the vesicles are capable of binding antigenic peptides and of inducing a specific cytotoxic (CTL) or helper T lymphocyte response, respectively. MHC class II molecules of these vesicles are also able to deliver superantigens to activate T Cells. Therefore, these vesicles can be used as an immune intervention method to treat different diseases basing on their various bioactive molecules.

Unexpectedly, the present invention demonstrates that vesicles derived from T cells express high amounts of MHC class I molecules (e.g., with a total amount at the same order as that of dendritic cells-derived vesicles from the same amount PBMC), while T cells are not considered as professional antigen-presenting cells.

Applicants have also provided evidence that these vesicles can be loaded with Class I peptides and are able to transfer the complexes of Class I/peptide to APCs, stimulating activation of specific T cells. This is particularly surprising since T cells themselves (from which the vesicles derive) are not antigen presenting cells.

The present application also demonstrates that the vesicles can be induced to express new molecules via efficient transfection of the producing T cells. This is particularly advantageous since other cell types such as dendritic cells, are difficult to transfect.

An other unexpected and advantageous aspect of this invention is that these vesicles can be produced in a much larger quantity from T cells than from other cell types. In particular, the invention shows that functional vesicles can be prepared from T cells that have been induced to proliferate and/or expand in culture.

This invention further shows that advantageous vesicles can be produced from established human T cell lines, thereby facilitating production of reproducible lots. Furthermore, established T cell lines can be transfected easily with appropriate nucleic acids (e.g., DNA vectors), allowing the expression of antigens or other proteins (including polypeptides) selectively enriched in exosomes.

The invention also shows that T cells lines can produce vesicles with significant different protein composition. As an example, Jurkart T cell line is shown to express very low amounts of HLA class I and II, high level of CD1c and significant level of CD1d. These vesicles could thus be used for CD1-specific antigen stimulation. Such T cell line could also be transfected with specific HLA class I or II haplotype to generate vesicles containing specific matched HLA haplotype, avoiding allogeneic response for therapeutic purpose.

The present invention thus provides novel biological products and compositions that can be used in the therapeutic or vaccination areas, particularly for delivering molecules to target cells. These products are particularly useful for producing antigen-specific immune response in vitro, ex vivo or in vivo.

Biological Preparation

According to the present invention, the vesicles and compositions can be produced using various biological preparations as a source of T lymphocytes. In particular, the biological preparation may comprise:
Freshly prepared T cells from various species, including PBMCs, blood sample, serum sample, plasma, bulk-cultured T cells and enriched T cell subsets such as, for example, CD8+ Cytotoxic/suppressor T cells, CD4+CD25− helper T cells, CD4+CD25+ regulatory T cells, λ/δ T cells and NKT cells;
T cells that can be maintained and expanded in vitro such as, for example, T cell lines, T cell clones, T hybridomas, and transformed T cells;
Malignant cells that are originated from T cells, for example T cell original leukemia cells;
T cells that are infected by viruses, or transfected by the genetic constructs encoding specific proteins.

The biological preparation may be treated to remove or expand particular cell sub-populations, particularly T lymphocytes specific for specific antigens or having a particular activity.

According to a particular, preferred embodiment, the biological preparation or the T cells are cultured under conditions triggering or increasing the yield of production of vesicles. Indeed, the present invention now demonstrates that particular treatments may be applied to improve vesicle production from T cells, such as:
Culturing the vesicles-producing cells with cytokines or other reagents to maintain, expand or change the properties of the T lymphocytes, for example, culture in the presence of IL1-a,β, IL-2, IL-7, IL-12, IL-15, IL-18, IL-4 and/or IL-13; and/or interferon gamma and/or antibodies against T cells surface markers, such as CD2, CD3, CD28, TcR, and/or soluble MHC class I or II tetramers and/or soluble CD1 tetramers.
Culturing the vesicles-producing cells with pharmaceutical reagents or particular treatments to induce maturation and/or activation of the cells, for example, in the presence of antigens, autologous or allogeneic APCs loaded with specific antigens or superantigens, mitogens (i.e., PHA), agrin, antibodies (such as anti-CD3 and anti-CD28 antibodies) or fragments thereof, reagents that trigger the activation of PKC (i.e., phorbol esters), cytoplasmic Ca⁺⁺ release (i.e., calcium ionophores), inhibition of phospatases (i.e., okadaic acid) etc.

In a preferred embodiment, the biological preparation comprises T lymphocytes that have been expanded and/or activated in culture.

In an other particular embodiment, the biological preparation comprises T lymphocytes that have been cultured in the presence of a TCR-activating agent.

In an other preferred embodiment, the biological preparation is a T cell line, particularly a T cell line which produces vesicles essentially devoid of endogenous HLA class I and II molecules.

In an other particular embodiment, the biological preparation is enriched for or comprises essentially a T cell subset, such as CD4+ T cells, CD8+ T cells, λδT cells, NKT cells, or for NK cells. Particularly preferred T cell subsets for delivering MHC Class I/II peptides are CD4+ T cells and CD8+ T cells. NK cells may also produce biologically active vesicles according to this invention.

In an other particular embodiment, the biological preparation comprises T lymphocytes that are specific for an antigen (a clonal population of T lymphocytes).

The biological preparation more preferably comprises at least 50% or more of T lymphocytes, more preferably 60% or more, even more preferably 70% or more. Most preferred methods of producing vesicles use biological preparations comprising essentially T lymphocytes, such as at least 90% T lymphocytes or more. In a typical embodiment, the biological preparation comprises at least 10E5 cells, more generally 10E6 cells at least. Furthermore, in a particular embodiment, the T cell are autologous with respect to the patient to be treated, although allogeneic or even xenogeneic cells may be used.

In a further particular embodiment, the T cells comprise a recombinant polynucleotide encoding a biologically active molecule. This embodiment will be disclosed in more details below.

Purification

The vesicles produced or released by T cells may be isolated and/or purified using several techniques. These include filtration, centrifugation, ion-chromatography, or concentration, either alone or in combinations.

A most preferred purification method comprises a step of density gradient centrifugation. An other preferred method comprises a step of ultrafiltration, either alone or coupled to a centrifugation step.

Suitable purification methods have been described in WO99/03499, WO00/44389 and WO01/82958, which are incorporated therein by reference.

Functionalization

The present application further demonstrates that T cell-derived vesicles may be functionalized to exhibit various biological activities. In particular, the vesicles may be modified so as to comprise any molecule of interest, such as proteins, polypeptides, peptides, lipids, glycolipids, nucleic acids, small drugs, saccharides, etc. This is particularly advantageous since the present application also provides evidence that these vesicles can efficiently deliver molecules to various target cells in vitro, ex vivo or in vivo, particularly to antigen-presenting cells (APCs).

As will be discussed below, the vesicles may be functionalized prior to, during or after their release or production by T cells. More precisely, they may be functionalized by direct loading of molecules, chimeric loading of molecules, or indirect loading (through modification of the producing T cells). Direct or chimeric loading may be performed on vesicles after their release.

The functionalizing molecule(s) may be present inside the vesicles, within their membrane, or at their surface. Molecules present within the cytosol may be various soluble factors, such as biologically active proteins or polypeptides, including cytokines, growth factors, hormones, RNA antisense, antibodies, tumor suppressor proteins, etc. Molecules may also be partially or wholly inserted within the vesicles membrane, such as receptors, sensors, etc. They may be inserted at the internal surface or the external surface of the membrane, or both (i.e., trans-membrane molecules). Molecules may also be associated at the surface of the vesicles, through various types of bonding, including covalent, electrostatic, hydrophobic or hydrogen bonds, etc. Molecules may be associated at the internal surface or the external surface of the membrane. Association may be made with specific markers or motifs present within the membrane, such as receptors, lipids, etc.

Direct Loading is a particular, preferred, embodiment of this invention. It is particularly suited to produce immunogenic vesicles loaded with specific antigenic peptides.

In a particular embodiment, antigenic molecules, for instance, may be associated at the surface of the vesicles by direct loading of antigen-presenting molecules, such as MHC Class I, Class II or CD1 molecules. In this regard, a further object of this invention is based on the unexpected enrichment of MHC molecules at the surface of T cell-derived vesicles. The inventors have now shown that such MHC molecules can be loaded directly with exogenous antigenic peptides (e.g., class I or Class II peptides). A particular object of this invention resides in a method of producing an immunogenic product, comprising:
providing T cell-derived vesicles, and
contacting said vesicles with an antigenic molecule under conditions allowing said molecule to interact with MHC complexes, thereby generating immunogenic products.

The invention also relates to a method of transferring antigenic peptides (e.g., MHC-binding peptides) or peptide/MHC complexes to APCs for inducing a specific T cell response, the method comprising:
a) Loading T-cell derived vesicles with antigenic peptides under conditions allowing binding to MHC molecules, and
b) Contacting said loaded vesicles with antigen-presenting cells, in vitro, ex vivo or in vivo, i.e., by direct administration of the vesicles to a subject.

Direct loading may be performed under various conditions as described in WO01/82958, incorporated therein by reference. In a preferred embodiment, the method comprises the step of subjecting the isolated or purified membrane vesicles to a selected acid medium or treatment prior to, during, or after contacting said vesicles with said immunogenic compound, so as to enable or facilitate loading thereof In this regard, the use of selected acid media or treatments allows to at least partially remove endogenous peptides or lipids associated at the surface of the vesicles and/or to facilitate the exchange of immunogenic compounds. In a further preferred embodiment, after contacting with the immunogenic compound, the vesicles are subjected to centrifugation, preferably density centrifugation, or diafiltration, to remove unbound immunogenic compound.

The immunogenic compound may be for instance any peptide or lipid, which are presented to an immune system in association with antigen-presenting molecules. The peptides may be class-I restricted peptides, class-II restricted peptides, either alone or in mixture or combination with other peptides, or even a peptide eluate of tumor cells. The invention is particularly suited for direct loading of class-I-restricted peptides. The lipids may be a microbial lipid, a microbial glycolipid or a lipid or glycolipid tumor antigen, either in isolated form or in various combination(s) or mixture(s).

In a preferred embodiment, the direct loading comprises (i) subjecting an isolated or purified membrane vesicle to a selected acid medium, (ii) contacting said isolated or purified membrane vesicle with a class I-restricted peptide under conditions allowing the peptide to complex with an HLA class I molecule at the surface of said membrane vesicle, and (iii) collecting the loaded membrane vesicle. The term “exogenous” means that the peptide is added to the composition.

In a further particular variant, the method comprises the steps of (i) subjecting an isolated or purified membrane vesicle to selected acid medium, (ii) contacting said isolated or purified membrane vesicle with a class I-restricted peptide in the presence of beta2-microglobulin, under conditions allowing the peptide to complex with an HLA class I molecule at the surface of said membrane vesicle, and (iii) collecting the loaded membrane vesicle. More preferably, step (i) comprises subjecting the isolated or purified membrane vesicles to an acid medium at a pH comprised between about 3 and 5.5, even more preferably between about 3.2 and 4.2, for less than 5 minutes. The direct loading approach in the presence of beta2-microglobulin is advantageous since it allows efficient loading even where only very small amounts of immunogenic compounds are available.

Where higher amounts of immunogenic compounds are available, no beta2-microglobulin is required and the method comprises the steps of (i) contacting an isolated or purified membrane vesicle with a class I-restricted immunogenic compound (e.g., peptide or lipid) in the absence of beta2-microglobulin, (ii) subjecting the mixture of (i) to a selected acid medium or treatment under conditions allowing the immunogenic compound to exchange with any endogenous compound for binding with an HLA class I molecule at the surface of said membrane vesicle, (iii) neutralizing the medium to stop the exchange and/or stabilize the complex formed in (ii) and (iv) collecting the loaded membrane vesicle. More preferably, step (ii) comprises subjecting the mixture to an acid medium at a pH comprised between about 4 and 5.5 for a period of time sufficient to produce an exchange between any endogenous molecule and the immunogenic compounds, for binding to the MHC complex.

Direct loading may also be implemented to functionalize the vesicles with larger antigens, such as viral proteins. In particular, viral proteins may interact directly with markers present at the surface of T cell-derived exosomes, such as the CD81. As a specific example, the hepatitis C virus (HCV) envelope glycoprotein can interact with CD81 at the surface of the vesicles, since CD81 is the receptor of hepatitis C and one of the major constituents of T cell-derived vesicles. A method of producing functionalized vesicles thus comprise contacting T cell-derived vesicles as disclosed above with a HCV envelope protein or a fragment thereof under conditions allowing said envelope protein or fragment thereof to bind CD81.

In this regard, a further object of this invention is a method of treating hepatitis C virus (HCV) infection in a subject, or of producing an immune response against HCV in a subject, the method comprising administering (e.g., injecting) to a subject in need thereof an effective amount of T-cell derived vesicles loaded with a HCV envelope protein or a CD81-binding fragment thereof. The envelope protein is more preferably the HCV envelope glycoprotein E2 or a CD81-binding fragment thereof. A more specific method comprises:
a) Preparing vesicles from T cells,
b) Loading the vesicles with a HCV envelope glycoprotein or a fragment thereof, typically E2 glycoprotein of HCV or a fragment thereof, or a chimeric construct incorporating E2 glycoprotein and additional immunogenic protein fragment of HCV and
c) injecting the loaded vesicles to a subject in need thereof, thereby causing or stimulating a specific immune response against HCV infection in said subject.

Alternatively, the loaded vesicles may be used to stimulate immune cells ex vivo or in vitro, said cells being administered to a subject.

The present invention also relates to a composition comprising T cell-derived vesicles loaded with a HCV glycoprotein or a fragment thereof, as well as to the use the said vesicles in vivo to treat or vaccinate against HCV infection. The invention also includes the use of T cell-derived vesicles to neutralize circulating HCV by binding the virus with CD81 and transfer of the virus to APCs, thereby inducing immune responses is against the HCV.

Chimeric Loading is an other particular, preferred embodiment of this invention. It is suited to produce vesicles loaded with any type of selected molecule, particularly proteins that may act on APCs or T cells in order to enhance the antigen presenting function of the vesicles. Chimeric loading may be accomplished through the use of a chimeric protein comprising a first domain having the ability to bind the membrane of the vesicles, and a second domain having selected activity. The first domain is preferably composed of lactadherin or E2 glycoprotein or a fragment thereof, typically lactadherin or a fragment thereof comprising the C1 and/or C2 domain thereof, or HCV E2 glycoprotein or a CD81-binding fragment thereof. Methods of producing such chimeric proteins have been disclosed in U.S. 60/313,159, which is incorporated therein by reference.

In particular, chimeric polypeptides or compounds can be prepared by genetic or chemical fusion. For the genetic fusion, the region of the chimeric gene coding for the polypeptide of interest may be fused upstream, downstream or at any internal domain junction of Lactadherin or E2 glycoprotein. Furthermore, the domains may be directly fused to each other, or separated by spacer regions that do not alter the properties of the chimeric polypeptide. Such spacer regions include cloning sites, cleavage sites, flexible domains, etc. In addition, the chimeric genetic construct may further comprise a leader signal sequence to favor secretion of the encoded chimeric polypeptide. For the chemical fusion, the partial or full-length lactadherin sequence may be selected or modified to present at its extremity a free reactive group such as thiol, amino, carboxyl group to cross-link a soluble polypeptide, a glycolipid or any small molecule. In a preferred embodiment, the Lactadherin construct encodes at least amino acids of the C1 domain and a Cysteine, providing a free thiol-residue for chemical cross-linking to other molecules. Crosslinking peptides, chemicals to SH groups can be achieved through well established methods (review G. T Hermanson (1996) Bioconjugate techniques San Diego Academic Press 785 pages).

The domain fused to lactadherin or E2 glycoprotein may be any polypeptide, protein, peptide, lipid, etc. It may also be a reactive domain capable of specifically binding to a modified selected molecule.

In a typical embodiment, the method of producing functionalized vesicles comprises providing T cell-derived vesicles and contacting said vesicles with a chimeric protein comprising a Lactadherin (or a fragment thereof comprising a C1 and/or C2 domain thereof) fused to a molecule of interest. As an example, the chimeric protein may comprise a lactadherin C1-C2-agrin molecule that can bind to the vesicles through the C1-C2 domain of lactadherin and increase the avidity of the antigen to prime a T cell response through the agrin moiety.

In an other typical embodiment, the method of producing functionalized vesicles comprises providing T cell-derived vesicles and contacting said vesicles with a chimeric protein comprising a HCV glycoprotein envelope (or a fragment or variant thereof comprising a CD81-binding domain) fused to a molecule of interest. As an example, the chimeric protein may comprise a HCV E2-agrin molecule that can bind to the vesicles through the CD81 marker and increase the avidity of the antigen to prime a T cell response through the agrin moiety.

Indirect Loading is an other particular, preferred embodiment of this invention. It is suited to produce vesicles loaded with various types of selected molecules. Indirect loading is based on a modification of the producing T cells. Such modification may be achieved by recombinant DNA technology (genetic modification) or by direct loading of T cells with antigenic molecules. In a particular embodiment, the T cells comprise a recombinant nucleic acid encoding a molecule of interest The nucleic acid may be a DNA or a RNA. It may be incorporated into various types of vectors, suitable to transfect or infect T cells, such as plasmids, viral vectors, naked DNA, etc. Upon transfection, the recombinant DNA is expressed in the cells and the expression product is delivered to vesicles. To further enhance targeting of the expressed molecules to vesicles, particular trafficking signals may be included in the recombinant nucleic acid, such as membrane-anchoring sequences, for instance. The encoded molecule may be an antigen, a peptide, a cytokine, growth factor, a ligand receptor, a receptor ligand, a TCR or a sub-unit thereof, etc.

In a particular embodiment, indirect loading is used to produce vesicles presenting defined TCR or MHC molecules. In particular, T cell lines may be transfected with a nucleic acid encoding specific MHC haplotype, thus producing immuno-competent vesicles from allogeneic T cell source. Such vesicles may then be further functionalized by direct loading of determined antigenic peptides, as described above.

These various methods allow the production of T-cell derived vesicles comprising discrete molecules of interest. These vesicles have improved biological properties and can be used to deliver antigens in vivo, to stimulate immune cells in vivo or in vitro, to produce an immune response, to deliver molecules to specific tissues, etc.

Production of an Immune Response

The present invention is particularly suited to produce, stimulate or regulate an immune response, particularly an antigen-specific immune response, such as a CTL response.

Indeed, while T cells are not considered as professional antigen-presenting cells, the present invention unexpectedly demonstrates that vesicles derived from T cells express high amounts of MHC class I molecules, that these vesicles can be loaded with Class I peptides, and that they are able to transfer the complexes of Class I/peptide to APCs, stimulating activation of specific T cells.

A particular aspect of this application resides in a method of producing or regulating an immune response in a subject, the method comprising administering to the subject an effective amount of a T-cell derived vesicle. A more preferred method is directed at producing or regulating an antigen-specific immune response in a subject, the method comprising administering to the subject an effective amount of a T-cell derived vesicle loaded with said antigen or an epitope thereof. These methods can be used as vaccines, to increase patient immunity to infections and tumors. The antigen may be a viral antigen, a bacterial antigen, a tumor antigen, a parasite, an autoantigen, etc.

This invention also provides that the specific TCRs present on the vesicles can be delivered to APCs, to induce an immune response for the specific epitopes of this TCR. In particular, vesicles of the present invention that are produced by a T cell clone, line, hybridoma or the malignant cells originated from T cells express a clonal TCR at their surface. Such clonal TCR may be used as an antigen, to produce specific immune responses. In particular, such vesicles may be used to deliver the special TCR (acting as the antigen) to APCs for inducing the immune responses specific for the epitopes of the TCR.

A particular object of this invention thus resides in the use vesicles produced from the cultured T cell clones/lines that recognize specific auto-antigens by their TCRs to deliver the carried TCRs to APCs for inducing an immune response against the harmful TCRs. This type of vesicles can be used as a TCR vaccine to treat autoimmune disorders.

An other particular object resides in the use vesicles produced from malignant cells that are originated from T cell, which T cells may have a cloned TCR, to deliver the carried TCRs as a specific tumor antigen to APC for inducing immune responses against the antigenic TCRs. This type of vesicles can be used as an idiotypic TCR vaccine to treat T cell leukemia.

A further object of this invention, based on the regulatory and effector molecules exhibited by the vesicles, is a method to induce the activation, inhibition or cytotoxicity of target cells through the bioactive membrane-bound proteins or proteins carried by the vesicles.

The vesicles may be used in any mammal, preferably in human subjects. They are typically administered by injection, e.g., intradermal, subcutaneous, intravenous, intra-arterial, intra-peritoneal, intramuscular, intra-tumoral (or in the vicinity of a tumor), etc. Repeated injections may be performed, if appropriate. The vesicles may be conditioned in various media, such as saline, isotonic, buffer, etc. The injected doses can range from about 1×10¹³ to about 1×10¹⁴ MHC class I molecules per dose, for instance.

Targeted Delivery of Molecules

A further object of this invention is based on the antigen specific TCRs present on the surface of the vesicles. The TCR may indeed be used as a targeting agent, to deliver any molecule specifically to target cells. The invention thus also relates to a method of targeting vesicles through their TCR component to target cells that express the antigens that can be recognized by the TCR.

Such methods may be used to specifically deliver bioactive molecules carried out by the vesicles to target cells that express the MHC-antigen complex that can be recognized by the TCR on the vesicle, as well as to specifically deliver the tracking or functional molecules that are loaded on the vesicles to the target cells that express the antigens that can be recognized by the TCR on the said vesicles.

Further aspects and advantages of the present invention will be disclosed in the following examples, which should be regarded as illustrative and not limiting the scope of protection. All references cited in this application are incorporated therein by reference.

### EXAMPLES

### Materials and Methods

1. Generation of Vesicles from Primary T Cells and T Cell Line.

1.1. Generation of Vesicles from Primary T Cells.

CD3+ T cells are enriched to 90% purity from the non-adherent cells of PBMC by removing non-T cells with a nylon wool column. The enriched CD3+ T cells are diluted to 4-5×10⁶/ml and cultured in the filtered AIMV medium using one of the following methods:
a. PHA at 1 μg/ml for 3 days,
b. PMA at 5 ng/ml plus ionomycin at 250 ng/ml for 3 days,
c. PHA at 1 μg/ml for 2 days. After replacing the medium with fresh and filtered AIMV medium, continuously culture fro another 4 days.

1.2. Generation of Vesicles from T Cell Line (Jurkat).

The Jurkat cells are diluted to 4-5×10⁶/ml and cultured in the filtered AIMV medium with PHA at 1 μg/ml for 4 days.

1.3. Purification of the Vesicles from T Cells

The vesicles produced according to examples 1.1 and 1.2 are purified from the T cell culture supernatant using the method disclosed in WO01/82958. Subsequently, they are concentrated to 150-200 times.

2. Characterization of the Vesicles

2.1. Phenotype of the Vesicles Measured by Aldehyde Bead Assay

The vesicles are conjugated to aldehyde polystyrene latex beads (Interface Dynamics Corporation) and stained with fluorescence labeled anti-CD antigen antibodies, before being subjected to FACS analysis.

2.2. Measurement of the Amount of Class I/II Molecules on the Vesicles by Quantitative FACS Analysis Using Aldehyde Bead Assay and Adsorption Elisa.

The ratio of Class I and Class II molecules at the surface of the vesicles is first measured by quantitative FACS analysis with aldehyde bead assay. The vesicles are is conjugated to the aldehyde beads and stained with unlabeled, mouse antibody against Class I/II plus fluorescence-labeled secondary antibody. The mean fluorescence intensity of the secondary antibody is compared with those on the aldehyde beads with known numbers of mouse Ig per bead. As a result, the numbers of Class I and Class II molecules at the surface of the vesicles per bead are generated, and the ratio of Class I and Class II molecules at the surface of the vesicles is deduced.

The absolute amount of Class II of the vesicles per microliter is measured by the adsorption Elisa, as described in WO01/82958.

The absolute amount of Class I molecules of the vesicles per microliter is calculated by the ratio of Class I versus Class II multiply absolute amount of Class II of the vesicles per microliter.

2.3. Functional Assay: SEE Assay

The vesicles are loaded with super antigen SEE and tested for their capacity of inducing IL-2 secretion of Jurkat T cells in the presence of Raji antigen presenting cells (WO01/82958).

3. Class I Peptide Loading

The MHC Class I molecules of the vesicles are directly loaded with biotin-labeled reference peptide. Binding of the reference peptide to the Class I molecule is demonstrated by the fluorescence signals generated from europium-avidin that bind to the biotin after the Class I molecules of the vesicle are captured on a plate.

The MHC Class I molecules of the said vesicles are directly loaded with a mixture of biotin-labeled reference peptide and target peptide. Binding of the target peptide to the Class I molecules is demonstrated by reduction of the binding of biotin-reference peptide, reflected by the reduced fluorescence signals from europium-avidin that binds to the biotin-labeled peptide as described in WO01/82958.

4. Biological Activity of the Vesicles Loaded with Mart-1 Class I Peptide.

The vesicles generated from HLA-A2+ T cells are directly loaded with Mart-1 peptide and tested for their capacity of inducing IFN-γ secretion of a Mart-1 specific T cell LT 11.

### Results

5. The Vesicles Enriched from Activated Primary T Cells Express T Cell-Specific Markers and Exosome Specific-Tetraspan Proteins.

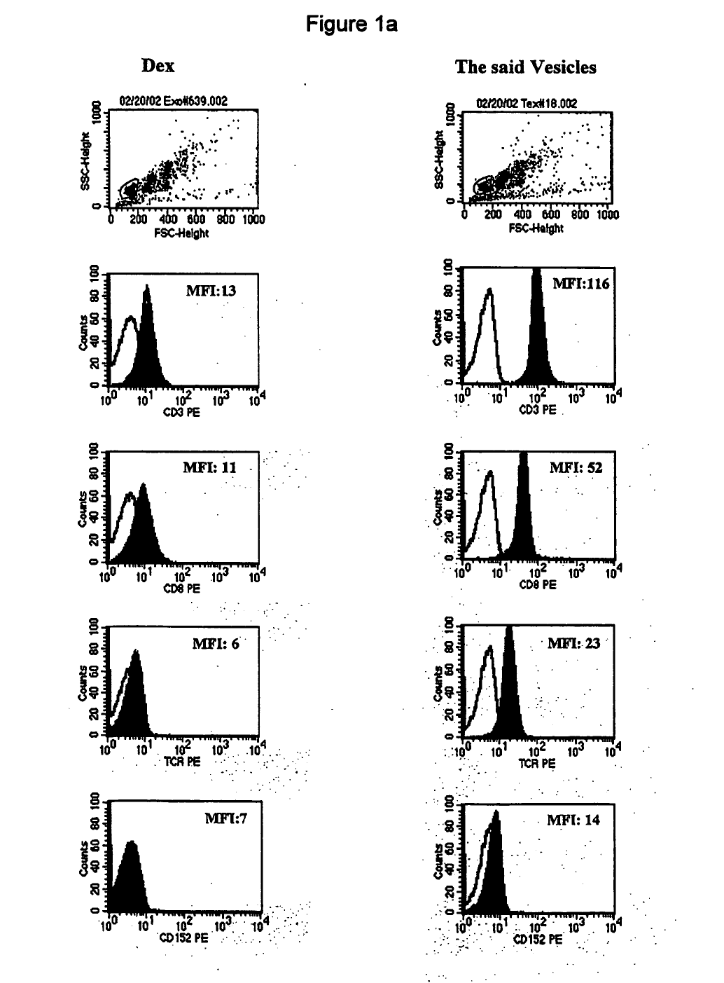

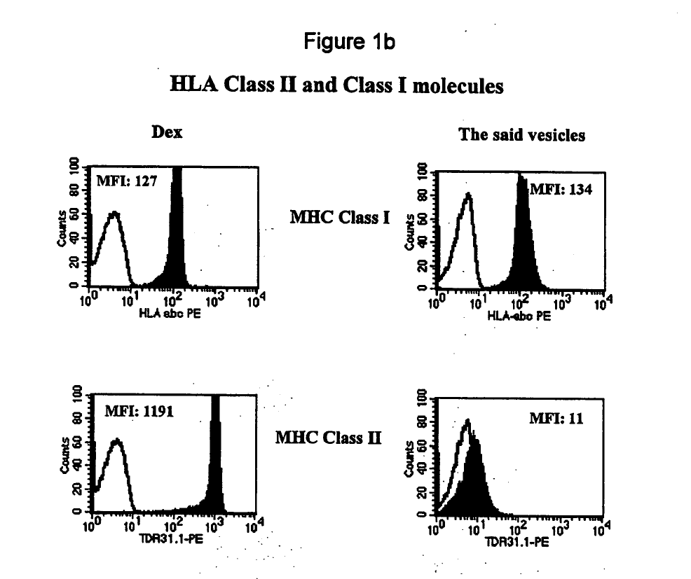

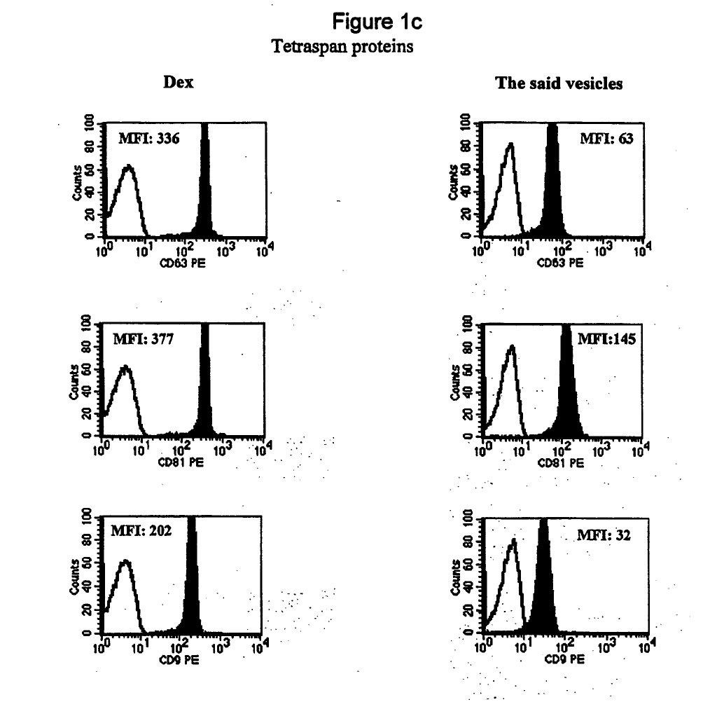

The phenotype of the membrane vesicles produced by PHA-activated T cells as disclosed in section 1. above has been analyzed by the aldehyde bead assay. The results are presented on FIG. 1

FIG. 1a shows that the vesicles derived from activated T cells express specific markers such as CD3, CD8, T cell receptor (TCR), and CD152. This is in contrast with vesicles derived from other cell types, such as dendritic cells (Dex), which essentially do not exhibit markers like CD3, CD8, TCR and CD152. The ratios of mean fluorescence intensity of the markers on T cell exosome and on Dex are 9.0, 4.7, 3.9, and 2.0 for CD3, CD8, TCR, and CD152 respectively.

FIG. 1b shows that the vesicles derived from activated T cells unexpectedly express more Class I than Class II (the ratio of mean fluorescence intensity between Class I and Class II is 12.2). This is in contrast with Dex, which express more Class II than Class I (the ratio of mean fluorescence intensity between Class I and Class II is 0.11).

FIG. 1c shows that the vesicles derived from activated T cells express tetraspan proteins like CD63, CD81, and CD9.

6. The Vesicles Generated Form Jurkat T Cell Line Express T Cell Specific Markers

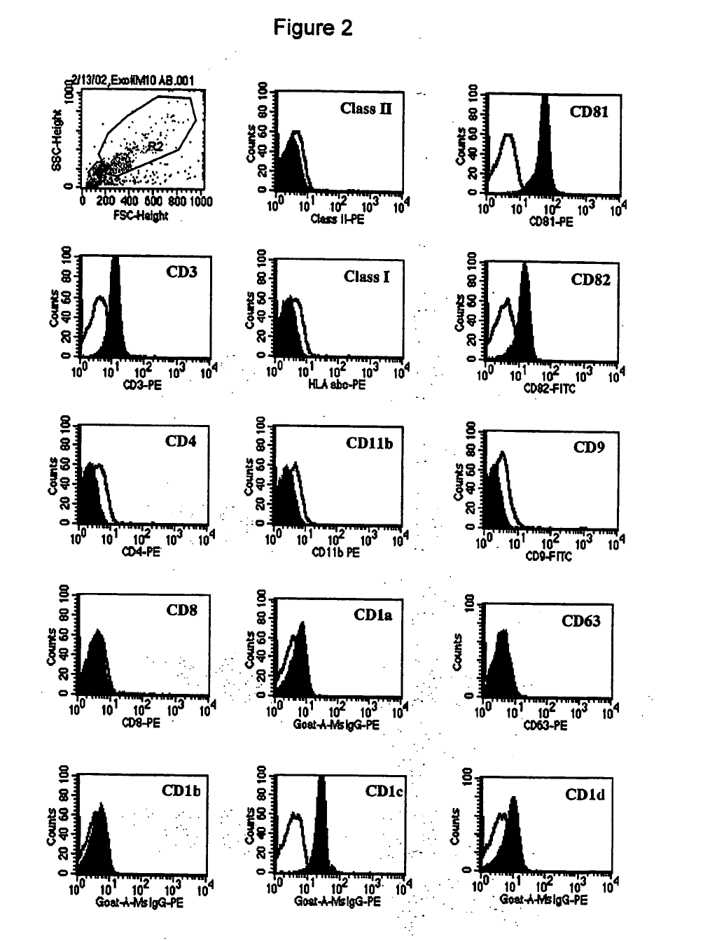

The phenotype of the membrane vesicles produced by PHA-activated Jurkart T cells as disclosed in section 1. above has been analyzed by the aldehyde bead assay. The results are presented on FIG. 2. Surprisingly, the vesicles express very low level of class I/II and high level of CD1c,d

7. The Total Class I Number of the Vesicles Produced from Activated T Cells is in the Same Order as that from Dentritic Cells.

Table 1 lists the total Class I number of the vesicles produced from activated T cells and Dex generated from three leukapacks. The Class I numbers are in as the same order for the said vesicles as for the Dex from each Leukapack without cell expansion.

It has been well documented that T cells are easily expandable up to 10,000 times by artificial antigen presenting cells (Maus M V et al 2000). They also can be immortalized (Hooijberg E. et al 2000, Kaltof K. 1998). Accordingly, the same amount of blood or Leukapack can be used to generate the vesicles carrying much higher total numbers of Class I molecules than Dex.

8. The Class II Molecules on the Vesicles from T Cells can be Loaded with Superantigen E (SEE) and Stimulate Jurkat T Cells to Secrete IL-2.

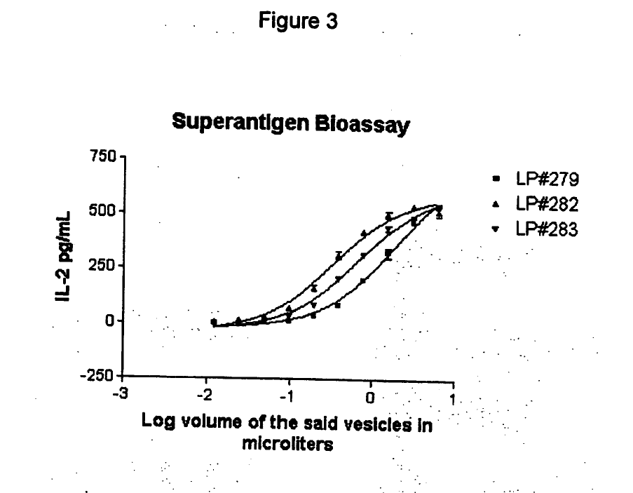

FIG. 3 shows that the vesicles generated from activated T cells of three leukapacks and loaded with superantigen SEE stimulate Jurkat cells to produce IL-2 in presence of APC. This demonstrates that such vesicles can transfer antigen HLA complexes to antigen presenting cells and make them fully functional.

9. The Class I Molecules on the Vesicles from T Cells can be Loaded with Biotin-Labeled Reference Peptide.

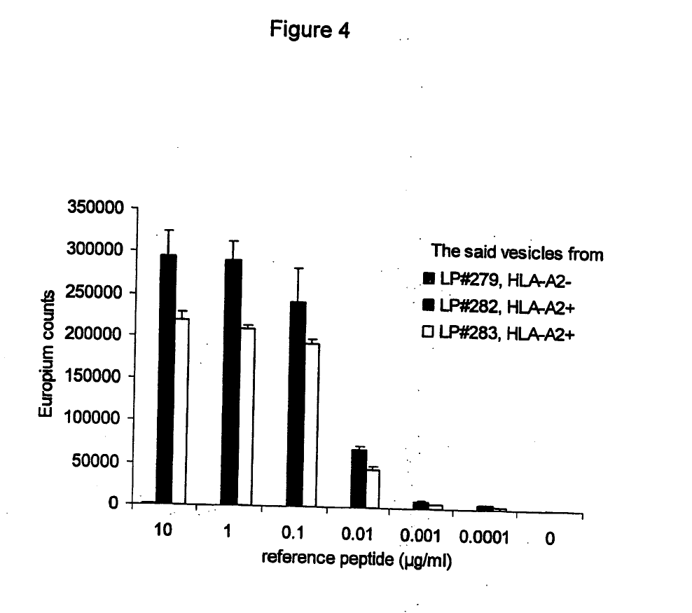

FIG. 4 shows that Class I molecules on the vesicles from T cells of HLA-A2+ leukapack, but not HLA-A2-Leukapack, can be directly loaded with HLA-A2-specific, biotin-labeled reference peptide at pH 4.2 and in the presence of β2 m. This provides evidence that the peptide loading is specific, because HLA restricted.

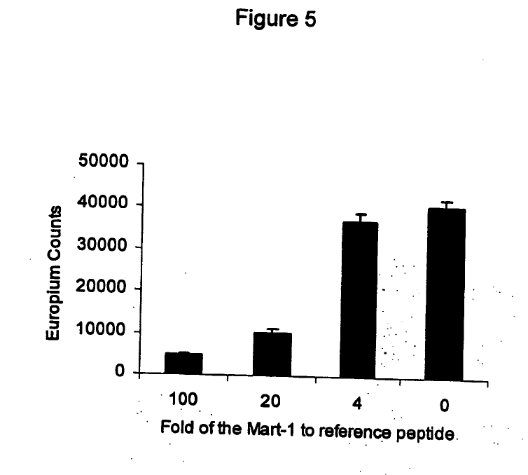

FIG. 5 shows that Class I molecules on the vesicles from HLA-A2+ T cells can be directly loaded with HLA-A2-specific peptide Mart-1 at pH 5.2 and in the absence of β2 m. The Mart-1 peptide competes off the binding of the biotin-labeled reference peptide. This shows that the Mart1 peptide can be loaded specifically to the MHC class I of the vesicles in a HLA restricted way.

10. The Class I Molecules on the Vesicles from HLA-A2+ T Cells Loaded with Mart-1 Peptide Induce Mart-1 Specific T Cells to Secret IFN-γ.

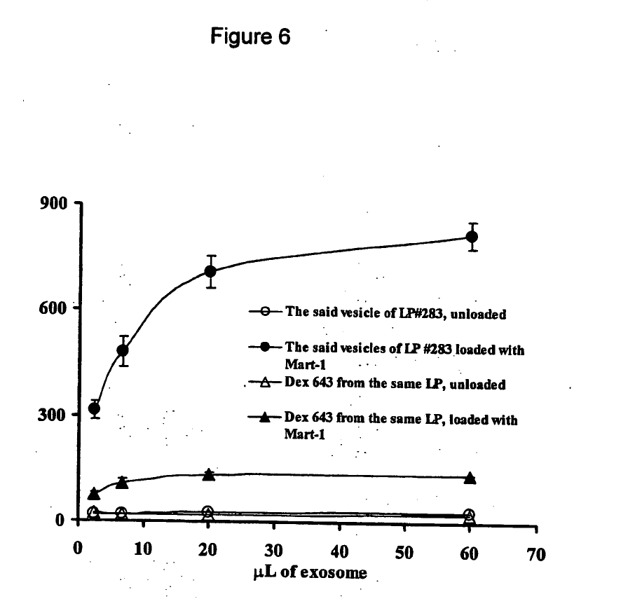

FIG. 6 clearly shows that the Mart-1 specific T cells LT11 respond to the stimulation of the said vesicles (HLA-A2+) loaded with the Mart-1 peptide and secrete IFN-γ. This demonstrates that the vesicles have been functionalized and are able to transfer the MHC class I peptide complex to the target APC.

TABLE 1
Comparison of the total Class I number of the T cell exosome and Dex
from leukopacks

| | The said vesicle | Dex |
|---|---|---|
| LP #279 | 0.87 × 10¹⁴ | 1.39 × 10¹⁴ |
| LP #282 | 0.25 × 10¹⁴ | 0.28 × 10¹⁴ |
| LP #283 | 2.2 × 10¹⁴ | 2.2 × 10¹⁴ |
| Mean | 1.1 × 10¹⁴ | 1.3 × 10¹⁴ |
| Standard Deviation | 0.97 × 10¹⁴ | 0.98 × 10¹⁴ |

### REFERENCES

Clayton, A., Court, J., Navabi, H., Adams, M., Mason, M. D., Hobot, J. A., Newman, G. R. and Jasani, B. Analysis of antigen presenting cell derived exosomes, based on immuno-magnetic isolation and flow cytometry. J Immunol Methods 247 163-74, 2001

Denzer, K, Keijmeer, M. J., Heijnen, H. F., Stoorvogel, W. and Geuze, H. J. Exosome: from internal vesicle of the multivesicular body to intercellular signaling device. J Cell Sci 113 Pt 19, 3365-74, 2000

Denzer, K, van Eijk, M., Kleijmeer, M. J., Jakobson, B., de Groot, C. and Geuze, H. J. Follicular dendritic cells carry MHC class II-expressing microvesicles at their surface. J Immunol 165, 1259-65, 2000

Escola, J. M., Kleijmeer, M. J., Stoorvogel, W., Griffith, J. M., Yoshie, O. and Geuze, H. J. Selective enrichment of tetraspan proteins on the internal vesicles of multivesicular endosomes and on exosomes secreted by human B-lymphocytes. J Biol Chem 273, 20121-7, 1998

Heijnen, H. F., Schiel, A. E., Fijnheer, R., Geuze, H. J. and Sixma, J. J. Activated platelets release two types of membrane vesicles: microvesicles by surface shedding and exosomes derived from exocytosis of multivesicular bodies and alpha-granules. Blood 94, 3791-9, 1999

Hooijberg E, Ruizendaal J J, Snijders P J, Kueter E W, Walboomers J M, Spits H. Immortalization of human CD8+ T cell clones by ectopic expression of telomerase reverse transcriptase. J Immunol 2000 Oct. 15; 165(8):4239-45

Kaltoft, K. Cytokine-driven immortalization of in vitro activated human T lymphocytes. CD28 expression correlates inversely with cell population doublings. Exp Clin Immunogenet 15, 84-9, 1998

Maus M V, Thomas A K, Leonard D G, Allman D, Addya K, Schlienger K, Riley J L, June C H. Ex vivo expansion of polyclonal and antigen-specific cytotoxic T lymphocytes by artificial APCs expressing ligands for the T-cell receptor, CD28 and 4-1BB. Nat Biotechnol. 2002 February 20(2):143-8.

Raposo, G., Nijman, H. W., Stoorvogel, W., Liejendekker, R, Harding, C. V., Melief, C. J. and Geuze, H. J. B lymphocytes secrete antigen-presenting vesicles. J Exp Med 183, 1161-72, 1996

Raposo, G., Tenza, D., Mecheri, S., Peronet, R., Bonnerot, C. and Desaymard, C. Accumulation of Major Histocompatibility Complex Class II Molecules in Mast Cell Secretory Granules and Their Release upon Degranulation. Mol Biol Cell 8, 2631-45, 1997

Thery, C., Regnault, A., Garin, J., Wolfers, J., Zitvogel, L., Ricciardi-Castagnoli, P., Raposo, G. and Amigorena, S. Molecular characterization of dendritic cell-derived exosomes. Selective accumulation of the heat shock protein hsc73. J Cell Biol 147, 599-610, 1999

Thery, C., Boussac, M., Veron, P., Ricciardi-Castagnoli, P., Raposo, G., Garin, J. and Amigorena, S. Proteomic analysis of dendritic cell-derived exosomes: a secreted subcellular compartment distinct from apoptotic vesicles. J Immunol 166, 7309-18, 2001

Van Niel, G., Raposo, G., Candalh, C., Boussac, M., Hershberg, R., Cerf-Bensussan, N. and Heyman, M. Intestinal epithelial cells secrete exosome-like vesicles. Gastroenterology 121, 337-49, 2001

Wolfers, J., Lozier, A., Raposo, G., Regnault, A., Thery, C., Masurier, C., Flament, C., Pouzieux, S., Faure, F., Tursz, T., Angevin, E., Amigorena, S. and Zitvogel, L. Tumor-derived exosomes are a source of shared tumor rejection antigens for CTL cross-priming. Nat Med 7, 297-303, 2001

Zitvogel, L., Regnault, A., Lozier, A., Wolfers, J., Flament, C., Tenza, D., Ricciardi-Castagnoli, P., Raposo, G. and Amigorena, S. Eradication of established murine tumors using a novel cell-free vaccine: dendritic cell-derived exosomes. Nat Med 4, 594-600, 1998

1-31. (canceled)

32. A method of producing lipid vesicles, wherein the method comprises:
a) culturing a biological preparation comprising T lymphocytes under conditions allowing the release of membrane vesicles from T lymphocytes, and
b) collecting or purifying vesicles produced in a),
and wherein said lymphocytes or vesicles are functionalized to express a selected molecule.

33. The method of claim 32, wherein said T lymphocytes are expanded and activated in culture.

34. The method of claim 33, wherein the T cells are cultured in the presence of a TCR-activating agent.

35. The method of claim 33, wherein said T lymphocytes are cultured in the presence of a cytokine, mitogen or calcium ionophore.

36. The method of claim 32, wherein said biological preparation comprises peripheral blood T cells, a T cell line, a T cell clone, a hybridoma or malignant T cells.

37. The method of claim 32, wherein the biological preparation is enriched for a T cell subset.

38. The method of claim 32, wherein the biological preparation is enriched for or comprises essentially NKT cells, NK cells, γδT cells, CD4+ cells or CD8+ cells.

39. The method of claim 32, wherein the biological preparation is a T cell line.

40. The method of claim 32, wherein the T cells comprise or have been transfected with a recombinant polynucleotide encoding a biologically active molecule.

41. The method of claim 32, wherein funtionalization of the vesicles is performed by direct loading of the vesicles with an antigenic molecule.

42. The method of claim 32, wherein funtionalization of the vesicles is performed by chimeric loading of the vesicles with a chimeric molecule comprising an active portion fused to a lactadherin or E2 glycoprotein, or to a fragment or variant thereof.

43. The method of claim 32, wherein the vesicles are collected or purified by filtration, centrifugation, ion-chromatography, or concentration, either alone or in combinations.

44. A method of producing immunogenic vesicles, the method comprising:
a) culturing a biological preparation comprising T lymphocytes under conditions allowing the release of membrane vesicles from T lymphocytes,
b) collecting or purifying vesicles produced in a), and
c) contacting said vesicles with an antigenic molecule under conditions allowing the molecule to bind said vesicles, so as to produce immunogenic vesicles.

45. The method of claim 44, wherein the antigenic molecule is a peptide, protein, lipid or a glycolipid.

46. The method of claim 44, wherein the antigenic molecule comprises a HCV envelope glycoprotein or a CD81-binding fragment thereof.

47. The method of claim 44, wherein the molecule is a chimeric protein comprising a polypeptide fused to lactadherin or to a HCV glycoprotein, or to a variant or fragment thereof.

48. A method of producing functionalized vesicles of claim 32, comprising:
a) culturing a biological preparation comprising T lymphocytes under conditions allowing the release of membrane vesicles from T lymphocytes, the biological preparation comprising T lymphocytes containing a recombinant nucleic acid encoding a selected molecule, and
b) collecting or purifying vesicles produced in a), said vesicles or a some of them at least comprising said selected molecule.

49. A method of producing vesicles according to claim 32, comprising:
a) culturing a clonal or idiotypic population of T lymphocytes having a determined T cell receptor under conditions allowing the release of membrane vesicles from T lymphocytes, and
b) collecting or purifying vesicles produced in a), said vesicles expressing at their surface said specific T cell receptor.

50. A method of producing vesicles according to claim 32, comprising:
a) culturing a T cell line under conditions allowing the release of membrane vesicles from T lymphocytes, and
b) collecting or purifying vesicles produced in a).

51. A method of producing lipid vesicles according to claim 32, the method comprising:
a) culturing a biological preparation comprising T lymphocytes in the presence of a T cell activating agent, allowing the release of membrane vesicles from T lymphocytes, and
b) collecting or purifying vesicles produced in a).

52. A method of producing a pharmaceutical composition, the method comprising:
a) culturing a biological preparation comprising T lymphocytes under conditions allowing the release of membrane vesicles from T lymphocytes,
b) collecting or purifying vesicles produced in a), and
c) conditioning said vesicles in a pharmaceutically acceptable carrier or excipient.

53. A pharmaceutical composition comprising a membrane vesicle and a pharmaceutically acceptable vehicle or excipient, wherein said vesicle is obtained from T lymphocytes.

54. The pharmaceutical composition of claim 53, wherein the vesicle comprises a selected molecule selected from a drug and an antigenic molecule.

55. A method of stimulating an immune response against an antigen in a subject, comprising administering to the subject an effective amount of a composition of claim 22.

56. A method of stimulating an immune response against an antigen in a subject, comprising:
a) culturing a biological preparation comprising T lymphocytes under conditions allowing the release of membrane vesicles from T lymphocytes,
b) collecting or purifying vesicles produced in a), wherein said vesicles are immunogenic,
c) conditioning said vesicles in a pharmaceutically acceptable carrier or excipient, and
d) administering the vesicles to a subject in an amount effective to stimulate an immune response.

57. A method of delivering an antigenic molecule to an antigen-presenting cell, comprising contacting in vitro or ex vivo antigen-presenting cells with a composition or an immunogenic vesicle according to claim 53, said vesicle comprising said antigenic molecule.

58. A method of stimulating dendritic cells, comprising contacting dendritic cells with a composition or an immunogenic vesicle according to claim 22.

59. A method of delivering a molecule to a target cell, comprising contacting said target cells with a composition or an immunogenic vesicle according to claim 53, said vesicle comprising said molecule.

60. A method of characterizing a preparation of vesicles derived from T cells, the method comprising:
isolating such vesicles from a biological preparation comprising T lymphocytes, and
determining the quantity or the quality of said vesicles by absorbing the same on a support and assessing the presence of specific markers at the surface of these vesicles.

61. A composition of claim 22 comprising an immunogenic membrane vesicle obtained from T lymphocytes and loaded with an antigenic molecule.

62. A composition of claim 61 comprising a vesicle derived from T lymphocyte and a HCV envelope glycoprotein or a CD81-binding fragment thereof.
